(19) United States
(12) Patent Application Publication
(10) Pub. No.: US 2023/0025560 A1
(43) Pub. Date: Jan. 26, 2023

(54) PROCESS FOR THE PRODUCTION OF MYCOPROTEIN

(71) Applicant: 3F BIO LTD, Glasgow (GB)

(72) Inventors: David Sparkes, Glasgow (GB); Robert James Taylor Laird, Glasgow (GB); Paul Andrew Hudman, Glasgow (GB)

(21) Appl. No.: 17/785,979

(22) PCT Filed: Dec. 18, 2020
(86) PCT No.: PCT/GB2020/053313

§ 371 Date: Jun. 16, 2022

(30) Foreign Application Priority Data
Dec. 20, 2019 (GB) 1919079.2

(51) Int. Cl. C12P 21/00; C07K 14/37

(52) U.S. Cl. CPC C12P 21/00; C07K 14/37

(57) ABSTRACT

There is described a continuous process for producing and isolating mycoprotein. The process may comprise the steps of: providing a fermentation media suitable for producing mycoprotein; introducing the fermentation media to a first fermentation vessel; fermenting the fermentation media to obtain a mixture comprising mycoprotein and partially spent fermentation media; isolating at least part of the partially spent fermentation media from the mixture comprising mycoprotein and partially spent fermentation media; and reintroducing at least a portion of the isolated partially spent fermentation media into the first fermentation vessel. Also described is mycoprotein obtained from the process.

### FIELD OF INVENTION

The present invention relates to a process for producing and isolating mycoprotein. In particular, the present invention relates to an efficient, low energy and cost-effective process for producing and isolating mycoprotein.

### BACKGROUND

Mycoprotein is a form of single-cell protein that is typically used as a food product or ingredient. It is conventionally produced by aerobic fermentation of a carbohydrate source using filamentous fungi, such as Fusarium venenatum.

GB2137226A describes a process for producing mycoprotein by continuous aerobic fermentation using Fusarium graminearum in a culture medium containing all necessary growth promoting nutrient substances. After the mycoprotein is grown by aerobic fermentation, a heat treatment step is required to reduce the content of nucleic acid, such as RNA, present in the mycoprotein product.

GB1440642A describes a method used to reduce the content of RNA in a mycoprotein product. A heat treatment step is performed on material that has been harvested by filtration, washed and then resuspended in water.

Although mycoprotein is a popular meat substitute, the production of mycoprotein is expensive. The high cost is associated with the use of refined feedstock (typically glucose syrup), high water usage, high energy costs associated with aerobic fermentation, and high plant operating costs.

WO 2016/063053 describes a process for the coproduction of mycoprotein and ethanol. In particular, mycoprotein is produced by aerobic fermentation of Fusarium species. The fermentation broth undergoes a heat treatment step to reduce RNA content and is then separated to provide mycoprotein paste and spent mycoprotein fermentation liquor. The spent mycoprotein fermentation liquor is then fed into an anaerobic fermentation process to provide ethanol.

However, the process described in WO 2016/063053 requires control of the growth conditions and substrate used to obtain the mycoprotein or ethanol in order to achieve the required mycoprotein to ethanol ratio. Furthermore, the heat treatment step in WO 2016/063053 can result in nutrient component interactions, such as Maillard reactions, which could negatively impact the quality and consistency of the fermentation liquor added to the anaerobic fermentation process.

Additionally, the heat treatment step in typical mycoprotein production processes have high energy costs and high processing times.

Therefore, it is an object of the present invention to provide an efficient, low energy and cost effective process for obtaining mycoprotein.

It is a further object of the invention to mitigate at least some of the disadvantages of the prior art. Further objects of the invention will be apparent from reading the following.

### DISCLOSURE OF INVENTION

According to the first aspect of the invention, there is provided a continuous process for producing and isolating mycoprotein, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) isolating at least part of the partially spent fermentation media from the mixture comprising mycoprotein and partially spent fermentation media; and
(v) reintroducing at least a portion of the isolated partially spent fermentation media into the first fermentation vessel.

By the term “media” is meant a solid, liquid or semi-solid designed to support the growth of microorganisms.

By the term “fermentation media” is meant media suitable for fermentation. For example, media comprising the components required to support the growth of microorganisms used for fermentation.

The fermentation media may comprise a carbohydrate suitable for producing mycoprotein, optionally wherein the carbohydrate is a sugar, optionally wherein the carbohydrate is glucose, sucrose or a source thereof. The carbohydrate may be glucose.

The fermentation media may be an aqueous fermentable broth suitable for producing mycoprotein.

The fermentation media may comprise water, a carbohydrate, a source of nitrogen and nutrients. The nutrients may be suitable for producing mycoprotein. The nutrients may be selected from one or more of the group consisting of: salts, vitamins and trace metals.

The salts may be selected from one or more of the group consisting of: potassium sulphate, potassium phosphate, magnesium sulphate, manganese chloride, calcium acetate, calcium chloride, iron sulphate, iron chloride, zinc sulphate, zinc chloride, copper sulphate, copper chloride, cobalt chloride, ammonium chloride, sodium molybdate, ammonium hydroxide, ammonium phosphate and choline salts.

By the term “partially spent fermentation media” is meant media that has undergone fermentation. The partially spent media may comprise at least a portion of the carbohydrate and/or nutrients from the original fermentation media. The carbohydrate may be a sugar, optionally the carbohydrate may be glucose, sucrose or a source thereof.

After the portion of the isolated partially spent fermentation media is reintroduced into the first fermentation vessel, the fermentation step may comprise fermenting the fermentation media and the portion of the isolated partially spent media to obtain a mixture comprising mycoprotein and partially spent fermentation media. After the portion of the isolated partially spent fermentation media is reintroduced into the first fermentation vessel, the fermentation step may comprise fermenting the carbohydrate from both the fermentation media and the portion of the isolated partially spent media to obtain a mixture comprising mycoprotein and partially spent fermentation media.

The fermentation media may be original fermentation media. The fermentation media may be a first fermentation media. The first, or original, fermentation media may comprise fresh, or new, media components that have not previously been subjected to the fermentation process (i.e., it is not partially spent fermentation media).

When partially spent fermentation media is combined with original fermentation media, the so-formed mixture of partially spent fermentation media and original fermentation media can be referred to as continuous fermentation media.

The continuous process may comprise:
(i) providing an original fermentation media suitable for producing mycoprotein;
(ii) introducing the original fermentation media to a first fermentation vessel;
(iii) fermenting the original fermentation media to obtain a mixture comprising mycoprotein and partially spent fermentation media;
(iv) isolating at least part of the partially spent fermentation media from the mixture comprising mycoprotein and partially spent fermentation media; and
(v) reintroducing at least a portion of the isolated partially spent fermentation media into the first fermentation vessel;
   wherein after the portion of the isolated partially spent fermentation media is reintroduced into the first fermentation vessel, the fermentation step may comprise fermenting the original fermentation media and the portion of the isolated partially spent media.

The carbohydrate in the fermentation media prior to fermentation may be in excess.

At least one of: (a) the provision of fermentation media after the reintroduction of a portion of the isolated partially spent fermentation media; and (b) the reintroduction of least a portion of the isolated partially spent fermentation media, may be configured to maintain an excess of carbohydrate prior to fermentation.

Maintaining an excess of carbohydrate prior to fermentation may comprise: (a) determining the concentration of carbohydrate in the partially spent fermentation media; and (b) adjusting the fermentation media introduced to the first fermentation vessel to maintain an excess of carbohydrate prior to fermentation, optionally wherein adjusting the fermentation media comprises reducing at least one of: the amount of fermentation media provided; and the concentration of carbohydrate therein.

The concentration of carbohydrate in the portion of the isolated partially spent fermentation media may be lower than the concentration of carbohydrate in the fermentation media prior to fermentation. The concentration of carbohydrate in the fermentation media prior to fermentation may be from approximately 15 g/L to approximately 90 g/L, optionally approximately 15 g/L to approximately 44 g/L, optionally approximately 15 g/L to approximately 33 g/L, optionally approximately 33 g/L to approximately 44 g/L, optionally approximately 16.5 g/L to approximately 49.5 g/L, optionally approximately 33 g/L. The concentration of carbohydrate in the fermentation media prior to fermentation may be at least 15 g/L. The concentration of carbohydrate in the portion of the isolated partially spent fermentation media that is reintroduced into the first fermentation vessel may be less than approximately 90 g/L, optionally from approximately 0.1 g/L to approximately 89.9 g/L, optionally less than approximately 33 g/L, optionally from approximately 0.1 g/L to approximately 32.9 g/L.

The concentration of carbohydrate in the fermentation media prior to fermentation may be 33 g/L. The concentration of carbohydrate in the portion of the isolated partially spent fermentation media that is reintroduced into the first fermentation vessel may be less than 33 g/L. At least one of: (a) the provision of the original, or first, fermentation media after the reintroduction of a portion of the isolated partially spent fermentation media; and (b) the reintroduction of least a portion of the isolated partially spent fermentation media, may be configured to maintain a carbohydrate concentration of at least 15 g/L, optionally 33 g/L in the fermentation media prior to fermentation.

The nutrients in the fermentation media prior to fermentation may be in excess and/or at a pre-determined concentration.

At least one of: (a) the provision of fermentation media after the reintroduction of a portion of the isolated partially spent fermentation media; and (b) the reintroduction of least a portion of the isolated partially spent fermentation media, may be configured to maintain an excess and/or a pre-determined concentration of nutrients prior to fermentation.

Maintaining an excess and/or a pre-determined concentration of nutrients prior to fermentation may comprise: (a) determining the concentration of one or more nutrients in the partially spent fermentation media; and (b) adjusting the fermentation media introduced to the first fermentation vessel to maintain an excess and/or a pre-determined concentration of nutrients prior to fermentation, optionally wherein adjusting the fermentation media comprises reducing at least one of: the amount of fermentation media provided; and the concentration of nutrients therein.

The concentration of nutrients in the portion of the isolated partially spent fermentation media may be lower than the concentration of nutrients in the fermentation media prior to fermentation.

When the nutrient is potassium, optionally potassium sulphate, the concentration may be from approximately 1 g/L to approximately 3 g/L, typically approximately 1.5 g/L to approximately 2.5 g/L, more typically approximately 2 g/L.

When the nutrient is magnesium, optionally magnesium sulphate, the concentration may be from approximately 0.45 g/L to approximately 1.35 g/L, typically approximately 0.68 g/L to approximately 1.13 g/L, more typically approximately 0.9 g/L.

When the nutrient is calcium, optionally calcium acetate, the concentration may be from approximately 0.1 g/L to approximately 0.3 g/L, typically approximately 0.15 g/L to approximately 0.25 g/L, more typically approximately 0.2 g/L.

When the nutrient is phosphoric acid, optionally 85% phosphoric acid, the concentration may be from approximately 0.575 g/L to approximately 1.725 g/L, typically approximately 0.86 g/L to approximately 1.44 g/L, more typically approximately 1.15 g/L.

When the nutrient is iron, optionally iron sulphate, the concentration may be from approximately 0.0025 g/L to approximately 0.0075 g/L, typically approximately 0.004 g/L to approximately 0.006 g/L, more typically approximately 0.005 g/L.

When the nutrient is zinc, optionally zinc sulphate, the concentration may be from approximately 0.0125 g/L to approximately 0.0375 g/L, typically approximately 0.019 g/L to approximately 0.031 g/L, more typically approximately 0.025 g/L.

When the nutrient is manganese, optionally manganese sulphate, the concentration may be from approximately 0.01 g/L to approximately 0.03 g/L, typically approximately 0.015 g/L to approximately 0.025 g/L, more typically approximately 0.02 g/L.

When the nutrient is copper, optionally copper sulphate, the concentration may be from approximately 0.00125 g/L to approximately 0.00375 g/L, typically approximately 0.0019 g/L to approximately 0.0031 g/L, more typically approximately 0.0025 g/L.

When the nutrient is biotin, the concentration may be from approximately 0.0000125 g/L to approximately 0.0000375 g/L, typically approximately 0.0019 g/L to approximately 0.000031 g/L, more typically approximately 0.000025 g/L.

When the nutrient is choline, optionally choline hydrochloride, the concentration may be from approximately 0.0435 g/L to approximately 0.1305 g/L, typically approximately 0.065 g/L to approximately 0.109 g/L, more typically approximately 0.087 g/L.

In the ranges and amounts given herein it will be understood that the different values given can be combined to provide different ranges and amounts. For example, where the ranges and amounts are given as 1 g/L to 3 g/L, typically 1.5 g/L to 2.5 g/L, more typically 2 g/L, this also includes 1 g/L to 2.5 g/L, 1 g/L to 2 g/L, 1 g/L to 1.5 g/L, 1.5 g/L to 3 g/L, 1.5 g/L to 2 g/L, 2 g/L to 3 g/L, 2 g/L to 2.5 g/L, 2.5 g/L to 3 g/L and/or any other combination of values, and/or the individual values of 1 g/L, 1.5 g/L, 2 g/L, 2.5 g/L and 3 g/L.

The specific growth rate of the mycoprotein may be from between approximately 0.17 h⁻¹ and approximately 0.2 h⁻¹.

The first fermentation vessel may be an aerobic fermentation vessel.

The first fermentation vessel may be a first aerobic fermentation vessel. The process may comprise the step of introducing the fermentation media to one or more aerobic fermentation vessels. The process may comprise the step of introducing the fermentation media to a first aerobic fermentation vessel and a second aerobic fermentation vessel.

The fermentation media may be fermented with a microorganism to obtain a mixture comprising mycoprotein and partially spent fermentation media, optionally wherein the microorganism is filamentous fungi, optionally wherein the filamentous fungi is selected from one or more of the group consisting of Aspergillus species, Rhizopus species and Fusarium species. The microorganism may be Fusarium venenatum.

Mycoprotein may be produced by aerobic fermentation using filamentous fungi. The filamentous fungi may be selected from one or more of the group consisting of Aspergillus species, Rhizopus species and Fusarium species. The filamentous fungi may be Fusarium venenatum. Mycoprotein may be produced by aerobic fermentation using Fusarium venenatum.

The process may comprise the additional step of removing the mixture comprising mycoprotein and partially spent fermentation media from the first fermentation vessel after the step of fermenting the fermentation media to obtain a mixture comprising mycoprotein and partially spent fermentation media.

The process may comprise the additional step of heating the mixture comprising mycoprotein and partially spent fermentation media.

The step of heating the mixture comprising mycoprotein and partially spent fermentation media may be after the step of isolating at least part of the partially spent fermentation media from the mixture.

The step of heating the mixture comprising mycoprotein and partially spent fermentation media may be after the step of reintroducing at least a portion of the isolated partially spent fermentation media into the first fermentation vessel.

The step of heating the mixture comprising mycoprotein and partially spent fermentation media may be before the step of isolating at least part of the partially spent fermentation media from the mixture.

The step of isolating at least part of the partially spent fermentation media from the mixture may comprise a first isolation step and a second isolation step.

The first isolation step may be before the step of heating the mixture comprising mycoprotein and partially spent fermentation media and the second isolation step may be after the step of heating the mixture comprising mycoprotein and partially spent fermentation media.

The step of reintroducing at least a portion of the isolated partially spent fermentation media into the first fermentation vessel may comprise a first reintroduction step and a second reintroduction step.

The first reintroduction step may be after the first isolation step and before the step of heating the mixture comprising mycoprotein and partially spent fermentation media.

The second reintroduction step may be after the second isolation step.

The mycoprotein in the mixture may be a substantially solid phase and the partially spent fermentation media in the mixture may be a substantially liquid phase comprising nutrients and a carbohydrate. The step of isolating at least part of the partially spent fermentation media from the mixture comprising mycoprotein and partially spent fermentation media may comprise separating the substantially solid phase and the substantially liquid phase.

The step of isolating at least part of the partially spent fermentation media from the mixture comprising mycoprotein and partially spent fermentation media may comprise separating the substantially solid phase and the substantially liquid phase by centrifugation, optionally wherein the separation is by filtration.

By the term “substantially solid phase” is meant solid-rich phase. By the term “substantially liquid phase” is meant liquid-rich phase.

The centrifugation may be disc stack centrifugation. However, any suitable centrifugation means and/or apparatus may be used.

The filtration may be cross flow filtration. However, any suitable filtration means and/or apparatus may be used.

The isolated partially spent media may be centrate, filtrate, or the like. The liquid-rich phase may be centrate. The liquid-rich phase may be filtrate.

The isolated partially spent media may be an aqueous solution comprising water, nutrients and a carbohydrate.

The reintroduction of at least a portion of the partially spent fermentation media into the first fermentation vessel may be a recycle step. The reintroduction of at least a portion of the partially spent fermentation media into the first fermentation vessel may decrease the amount of fermentation media required for the process. The reintroduction of at least a portion of the partially spent fermentation media into the first fermentation vessel may decrease the amount of carbohydrate and/or water required for the process.

The process may comprise a sterilisation step after the isolation step and before the step of reintroducing at least a portion of the isolated partially spent fermentation media into the first fermentation vessel. The sterilisation step may be after the second isolation step and before the step of reintroducing at least a portion of the isolated partially spent fermentation media into the first fermentation vessel.

The sterilisation step may be a heat-sterilisation step or a filter-sterilisation step.

The process may comprise the additional step of producing and isolating ethanol.

The fermentation media may be obtained from a feedstock. The feedstock may be at least one of a starch-based feedstock and a sugar-based feedstock. The starch-based feedstock may be selected from one or more of the group consisting of a grain, cassava and potatoes. The feedstock may be a grain. The grain may be at least one of wheat, maize, buckwheat, rye, barley, millet and rice. The sugar-based feedstock may be selected from one or more of the group consisting of sugarcane, sugar beets and sweet sorghum. The feedstock may be sugarcane. The feedstock may be subjected to one or more of milling, grinding, gelatinisation, liquefaction and saccharification before the step of introducing the fermentation media to the first fermentation vessel. The fermentation media may be an aqueous fermentable broth comprising hydrolysed starch.

The process may comprise the additional step of introducing at least a portion of the isolated partially spent fermentation media into a second fermentation vessel. The second fermentation vessel may be an anaerobic fermentation vessel.

The step of introducing at least a portion of the isolated partially spent fermentation media into the second fermentation vessel may be after the step of reintroducing at least a portion of the isolated partially spent fermentation media into the first fermentation vessel.

The process may comprise the additional step of fermenting the at least a portion of the isolated partially spent fermentation media in the second fermentation vessel to obtain ethanol.

The at least a portion of the isolated partially spent fermentation media may be reintroduced into the first fermentation vessel and the remainder of the isolated partially spent fermentation media may be introduced into the second fermentation vessel.

At least a portion of the isolated partially spent fermentation media may be reintroduced into the first fermentation vessel and the remainder of the isolated partially spent fermentation media may introduced into the second fermentation vessel. Optionally, the remainder of the isolated partially spent fermentation media may be discharged as effluent.

The process may be operated with excess fermentation media components. The process may be operated with an excess of carbohydrate in the fermentation media, optionally wherein the carbohydrate is glucose.

According to a second aspect of the invention, there is provided mycoprotein obtainable, obtained or directly obtained by the process described in the first aspect.

According to a third aspect of the invention, there is provided a continuous process for producing and isolating mycoprotein, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) a first isolation step comprising isolating at least part of the partially spent fermentation media from the mixture comprising mycoprotein and partially spent fermentation media;
(v) reintroducing at least a portion of the isolated partially spent fermentation media into the first fermentation vessel;
(vi) heating the mixture comprising mycoprotein and partially spent fermentation media; and
(vii) a second isolation step comprising isolating at least part of the partially spent fermentation media from the mixture comprising mycoprotein and partially spent fermentation media.

The second isolation step may comprise isolating at least part of the partially spent fermentation media from the heat-treated mixture comprising mycoprotein and partially spent fermentation media.

According to a fourth aspect of the invention, there is provided mycoprotein obtainable, obtained or directly obtained by the process described in the third aspect.

According to a fifth aspect of the invention, there is provided a continuous process for producing and isolating mycoprotein, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) heating the mixture comprising mycoprotein and partially spent fermentation media;
(v) isolating at least part of the partially spent fermentation media from the mixture comprising mycoprotein and partially spent fermentation media; and
(vi) reintroducing at least a portion of the isolated partially spent fermentation media into the first fermentation vessel.

The process may comprise a sterilisation step after the isolation step and before the step of reintroducing at least a portion of the isolated partially spent fermentation media into the first fermentation vessel.

The sterilisation step may be a heat-sterilisation step or a filter-sterilisation step.

According to a sixth aspect of the invention, there is provided mycoprotein obtainable, obtained or directly obtained by the process described in the fifth aspect.

According to a seventh aspect of the invention, there is provided a continuous process for producing and isolating mycoprotein, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) a first isolation step comprising isolating at least part of the partially spent fermentation media from the mixture comprising mycoprotein and partially spent fermentation media;
(v) a first reintroduction step comprising reintroducing at least a portion of the isolated partially spent fermentation media into the first fermentation vessel;
(vi) heating the mixture comprising mycoprotein and partially spent fermentation media;
(vii) a second isolation step comprising isolating at least part of the partially spent fermentation media from the mixture comprising mycoprotein and partially spent fermentation media; and
(viii) a second reintroduction step comprising reintroducing at least a portion of the isolated partially spent fermentation media into the first fermentation vessel.

The second isolation step may comprise isolating at least part of the partially spent fermentation media from the heat-treated mixture comprising mycoprotein and partially spent fermentation media.

The second reintroduction step may comprise reintroducing at least a portion of the second isolated partially spent fermentation media into the first fermentation vessel.

According to an eight aspect of the invention, there is provided mycoprotein obtainable, obtained or directly obtained by the process described in the seventh aspect.

According to a ninth aspect of the invention, there is provided a recycling process, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) isolating at least part of the partially spent fermentation media from the mixture comprising mycoprotein and partially spent fermentation media; and
(v) recycling at least a portion of the isolated partially spent fermentation media into the first fermentation vessel.

The recycling process may be a process for recycling fermentation media.

According to a tenth aspect of the invention, there is provided mycoprotein obtainable, obtained or directly obtained by the process described in the ninth aspect.

According to an eleventh aspect of the invention, there is provided an integrated continuous process for producing and isolating mycoprotein and ethanol, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) heating the mixture comprising mycoprotein and partially spent fermentation media;
(v) isolating at least part of the partially spent fermentation media from the mixture comprising mycoprotein and partially spent fermentation media;
(vi) reintroducing at least a portion of the isolated partially spent fermentation media into the first fermentation vessel; and
(vii) introducing the remainder of the isolated partially spent fermentation media into a second fermentation vessel.

The process may comprise the additional step of fermenting the remainder of the isolated partially spent fermentation media in the second fermentation vessel to obtain ethanol.

The remainder of the isolated partially spent media may be fermented with a microorganism to obtain ethanol. The microorganism may an alcohol-producing microorganism. The microorganism may be Saccharomyces cerevisiae.

The remainder of the isolated partially spent media may be fermented with a portion of unfermented aqueous fermentable broth and a microorganism to obtain ethanol.

The second fermentation vessel may be an anaerobic fermentation vessel.

According to a twelfth aspect of the invention, there is provided mycoprotein obtainable, obtained or directly obtained by the process described in the eleventh aspect.

According to a thirteenth aspect of the invention, there is provided an integrated continuous process for producing and isolating mycoprotein and ethanol, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) a first isolation step comprising isolating at least part of the partially spent fermentation media from the mixture comprising mycoprotein and partially spent fermentation media;
(v) a first reintroduction step comprising reintroducing at least a portion of the isolated partially spent fermentation media into the first fermentation vessel;
(vi) heating the mixture comprising mycoprotein and partially spent fermentation media;
(vii) a second isolation step comprising isolating at least part of the partially spent fermentation media from the mixture comprising mycoprotein and partially spent fermentation media; and
(viii) introducing at least a portion of the isolated partially spent fermentation media into a second fermentation vessel.

According to a fourteenth aspect of the invention, there is provided mycoprotein obtainable, obtained or directly obtained by the process described in the thirteenth aspect.

The alternative features and different embodiments as described apply to each and every aspect and each and every embodiment thereof mutatis mutandis.

### BRIEF DESCRIPTION OF THE DRAWINGS

Embodiments of the invention will now be described, by way of example, with reference to the drawings, in which:

FIG. 1 is a flow diagram which illustrates a process in accordance with one embodiment of the invention;

FIG. 2 is a flow diagram which illustrates a process in accordance with a second embodiment of the invention; and

FIG. 3 is a flow diagram which illustrates a process in accordance with a third embodiment of the invention.

FIG. 4 is a flow diagram which illustrates a process in accordance with a fourth embodiment of the invention.

FIG. 5 shows images of the hyphal length of filaments in the mixture comprising mycoprotein and partially spent media obtained (a) before separation by disc stack centrifugation, (b) after a first separation by disc stack centrifugation, and (c) after a second example of a first separation by disc stack centrifugation. (Experiment 1A)

FIG. 6 shows the growth of mycoprotein (F. venenatum) on pure glucose in fermentation media comprising isolated partially spent fermentation media (isolated from a mixture comprising mycoprotein and partially spent fermentation media post-heat treatment) from a previous aerobic fermentation reaction. (Experiment 2)

FIG. 7 shows a correlation graph of the biomass concentration versus the glucose concentration in a continuous fermentation reaction with a fixed concentration and flow rate of glucose feed as the fermentation media. (Experiment 3)

FIG. 8 shows the effect of continuous recycling of isolated partially spent fermentation media on media nutrients (macronutrients). (Experiment 4)

FIG. 9 shows the effect of continuous recycling of isolated partially spent fermentation media on media nutrients (micronutrients). (Experiment 4)

### DETAILED DESCRIPTION

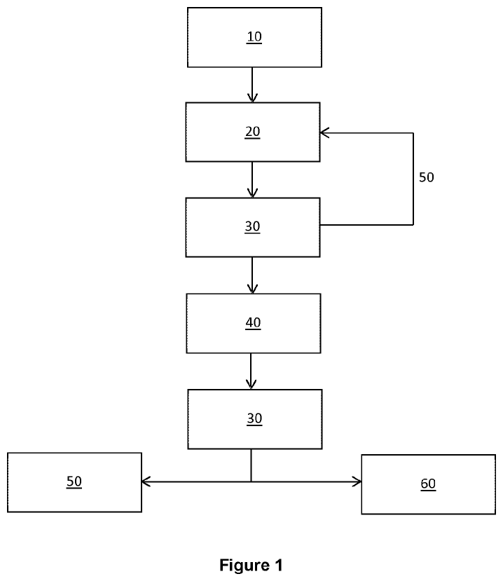

Referring to FIG. 1, there is shown a process for producing and isolating mycoprotein. A fermentation media 10 that is rich in glucose is added to the first fermentation vessel 20. The fermentation media 10 comprises water, a carbohydrate, a source of nitrogen and nutrients. The carbohydrate is typically glucose. The nutrients are typically selected from salts, vitamins and trace metals. The salts are typically selected from one or more of the group consisting of potassium sulphate, potassium phosphate, magnesium sulphate, manganese chloride, calcium acetate, calcium chloride, iron sulphate, iron chloride, zinc sulphate, zinc chloride, copper sulphate, copper chloride, cobalt chloride, ammonium chloride, sodium molybdate, ammonium hydroxide and ammonium phosphate. Other components that are optionally added to the fermentation media include, but are not limited to, biotin, choline and phosphoric acid. The fermentation media 10 is cooled to 30° C. and inoculated with a mycoprotein-producing microorganism. The mycoprotein-producing microorganism is a filamentous fungi, optionally from the Fusarium species, and is typically Fusarium venenatum.

Aerobic conditions are maintained by aerating and agitating the media.

The product of the aerobic fermentation is a mixture comprising mycoprotein 60 and partially spent fermentation media 50. The partially spent fermentation media 50 comprises nutrients and glucose from the fermentation media.

In one embodiment, as shown in FIG. 1, at least part of the partially spent fermentation media 50 is isolated 30 from the mixture comprising mycoprotein 60 and partially spent fermentation media 50 after aerobic fermentation. The isolation step 30 may be performed by any solid-liquid separation means and/or apparatus known in the art. For example, centrifugation, filtration, or the like. The mixture obtained from the aerobic fermentation comprises a solid-rich phase and a liquid-rich phase. The solid-rich phase substantially comprises the mycoprotein 60 and the liquid-rich phase substantially comprises the partially spent fermentation media 50. This isolation step is not 100% efficient. Therefore, at least part of the partially spent fermentation media 50 is isolated 30 from the mixture and the remaining mixture comprises mycoprotein 60 and partially spent fermentation media 50. At least a portion of the isolated partially spent fermentation media 50 is then reintroduced into the first fermentation vessel 20. After the isolation step 30, the mixture comprising mycoprotein 60 and partially spent fermentation media 50 will then continue through the rest of the mycoprotein production process.

The recycle of the isolated partially spent fermentation media 50 into the first fermentation vessel 20 replaces water, glucose and/or nutrient content in the fermentation media 10. Therefore, the volume of fermentation media 10 introduced to the first fermentation vessel 20 can be reduced. The reduction in the volume of water required for the fermentation media 10 will reduce operating costs because the water used in the aerobic fermentation process requires pre-treatment before being used in the fermentation media 10. This will also reduce the volume of waste effluent generated by the process. This reduction of water in both the fermentation media 10 and waste effluent significantly reduces the carbon footprint of the mycoprotein production process due to the large capacity plants (typically between 10,000 L to 700,000 L) that operate the mycoprotein production process.

By reintroducing the isolated partially spent fermentation media 50 into the first fermentation vessel 20 directly after aerobic fermentation, the isolated partially spent fermentation media 50 will still comprise the nutrients and glucose necessary for mycoprotein fermentation.

As shown in FIG. 1, after the isolation step 30 the mixture comprising mycoprotein 60 and partially spent media 50 is then subjected to a heat treatment step 40 in order to degrade nucleic acid, such as RNA, which may be present. The advantage of isolating 30 at least part of the partially spent fermentation media 50 from the mixture before heat treatment 40 is that the volume of the mixture that needs to undergo heat treatment 40 is significantly reduced. This results in decreased process operating costs due to decreased processing time and the reduced requirement for steam, electricity and waste treatment and disposal.

Following heat treatment 40, a second isolation step 30 is carried out to isolate at least part of the partially spent media 50 from the mixture comprising mycoprotein 60 and partially spent fermentation media 50.

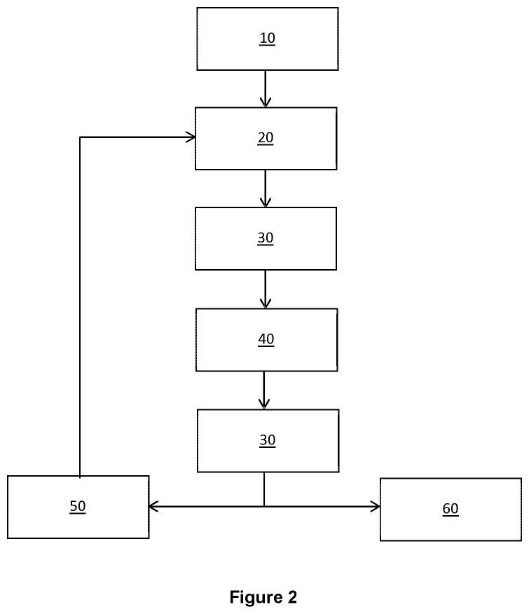

The mixture comprising mycoprotein 60 and partially spent fermentation media 50 is then subjected to further processing steps to provide isolated mycoprotein 60. The isolated partially spent fermentation media 50 from the second isolation step 30 can be disposed or discharged from the process as waste effluent or recycled in the process as described in FIGS. 2 to 4.

Referring to FIG. 2, there is shown a second embodiment of the invention, wherein at least a portion of the isolated partially spent fermentation media 50 obtained post-heat treatment 40 is recycled or reintroduced into the first fermentation vessel 20.

At this stage, an additional sterilisation step is required before the isolated partially spent fermentation media 50 is reintroduced to the first fermentation vessel 20. This is because, in the design of typical mycoprotein production plants, the second isolation step 30 is typically carried out by centrifugation in non-sterile conditions. Therefore, the isolated partially spent fermentation media 50 may be subjected to heat-sterilisation or filter-sterilisation before it is reintroduced to the first fermentation vessel 20. The heat-sterilisation may be carried out using a heat exchanger.

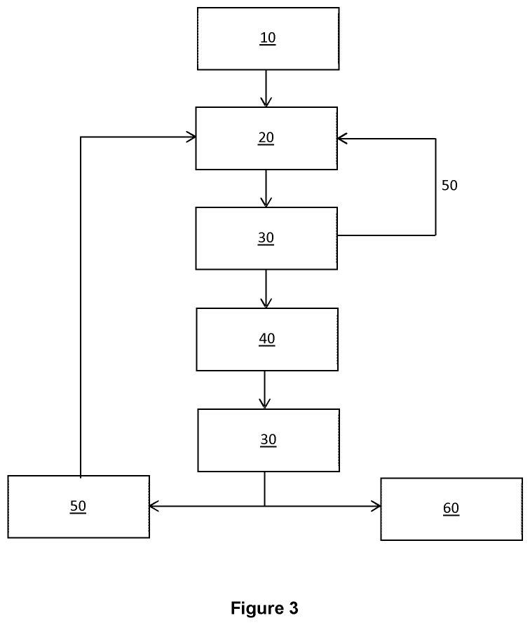

Referring to FIG. 3, there is shown a third embodiment of the invention, wherein at least a portion of the isolated partially spent fermentation media 50 obtained pre-heat treatment 40 and post-heat treatment 40 is recycled or reintroduced into the first fermentation vessel 20.

The reintroduction of the isolated partially spent fermentation media 50 after the first isolation step 30 does not require an additional sterilisation step because the process is sterile at this stage.

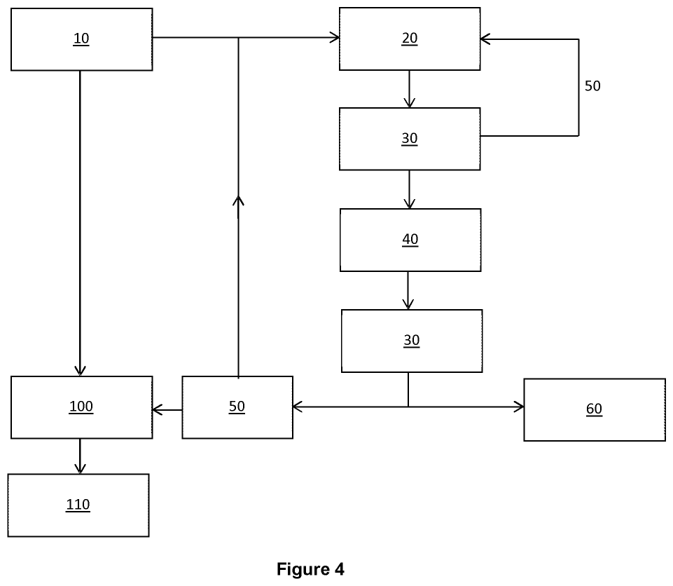

Referring to FIG. 4, there is shown a fourth embodiment of the invention, wherein the mycoprotein production process is integrated with an existing ethanol biorefinery. In FIG. 4, the fermentation media 10 for the mycoprotein production process is obtained from the ethanol production process.

In a typical ethanol biorefinery, biomass feedstock, such as wheat, maize or sugarcane, undergoes anaerobic fermentation to yield ethanol.

The feedstock, e.g., a grain that is rich in starch, such as wheat or maize, is milled or ground in a feedstock processing tank to generate a flour. The flour is added to a mash tank and mixed with water and enzymes, e.g., amylases, to generate a mash, which is then heated to hydrolyse the starch from the feedstock into fermentable sugars. The mash tank is heated in two stages; the mash is heated to 85° C. for two hours, the temperature is then lowered to 60° C. and maintained at 60° C. for four hours. The resulting hydrolysed mash, which is rich in glucose, is then used as part of the fermentation media 10 for the aerobic fermentation reaction.

A portion of the hydrolysed mash 10 is removed from the mash tank and provided to a first fermentation reaction vessel 20. The hydrolysed mash is optionally filter sterilised and subjected to a saccharification step prior to being added to the first fermentation reaction vessel 20. The filter sterilisation process involves centrifugation of the hydrolysed mash, followed by filtration, optionally using 0.2 μm filters. The liquid phase is then added to the first fermentation reaction vessel 20.

The hydrolysed mash is then mixed with at least one source of nitrogen, water and nutrients to generate a fermentation media 10 as described above.

In FIG. 4, at least a portion of the isolated partially spent fermentation media 50 obtained pre-heat treatment 40 and post-heat treatment 40 is recycled or reintroduced into the first fermentation vessel 20.

Additionally, a second portion of the isolated partially spent fermentation media 50 obtained post-heat treatment 40 is introduced to a second fermentation reaction vessel 100. Anaerobic fermentation reaction conditions are introduced to the second vessel 100 by lowering the temperature to 30° C. and inoculating the fermentation media with an alcohol-producing microorganism, such as Saccharomyces cerevisiae.

The anaerobic fermentation reaction yields a fermented mash comprising ethanol 110 and a residue, which is transferred to a distillation vessel. The distillation vessel is operated at 63° C. under vacuum to separate the bioethanol 110 from the fermentation residue. Carbon dioxide is generated as a co-product of the fermentation reaction.

The following experiments were performed to support the invention.

### Experiment 1A: Separation of Mycoprotein and Partially Spent Media Before Heat Treatment Step (12 L)

A fermentation media 10 is prepared by adding the nutrients outlined in Table 1 to 12 L of deionised water.

TABLE 1
Nutrient Composition of Fermentation Media

| | Concentration |
|---|---|
| Fermentation Media Component | (g/L) |
| Potassium sulphate (K₂SO₄) | 2 |
| Magnesium sulphate heptahydrate (MgSO₄•7H₂O) | 0.9 |
| Calcium Acetate (Ca(C₂H₃O₂)₂) | 0.2 |
| Phosphoric Acid (85%) | 1.15 (mL/L) |
| Iron (II) sulphate heptahydrate (FeSO₄•7H₂O) | 0.005 |
| Zinc sulphate heptahydrate | 0.025 |
| Manganese sulphate tetrahydrate (MnSO₄•4H₂O) | 0.02 |
| Copper sulphate heptahydrate (CuSO₄•7H₂O) | 0.0025 |

The media 10 is added to a first fermentation vessel 20 and sterilised by heating the fermentation vessel 20 using a heated water jacket. The temperature is maintained at 121° C. for 30 minutes. Before sterilisation, care is taken to carefully secure all connections in the first fermentation vessel 20; for example, all addition ports are secured using rubber septum and respective collar fittings.

After sterilisation, filter sterilised glucose (44 g/L), biotin (0.000025 g/L) and choline hydrochloride (0.087 g/L) are transferred into the first fermentation vessel 20 under aseptic conditions using a peristatic pump after the first fermentation vessel 20 is cooled down to an ambient temperature.

A dissolved oxygen (DO) probe is inserted into the fermentation vessel 20 before sterilisation. The probe is then calibrated after sterilisation. The DO probe is calibrated at a fermentation temperature of 30° C., with an air flow of 10 L/min (1 VVM (volume of air per volume of liquid per minute)) and stirring speed of 300 rpm using compressed air and nitrogen gas. Nitrogen gas is flushed through a sparger at a rate of 10 L/min to achieve 0% calibration of the DO probe. Similarly, compressed air is then sparged into the fermentation media 10 until saturation is achieved (i.e., a constant reading is observed) to allow 100% calibration. The air enters the first fermentation vessel 20 through a sterile inlet filter and sparger. Air escapes first through a condenser, to ensure there is no loss of media 10, and then through an exit filter. Thereafter, the pH of the fermentation media 10 is adjusted to pH 6.0 using a suitable base (in this example 35% Ammonium Hydroxide is used as the base).

Fermentation is initiated by adding 1 L of 1% w/v inoculum (Fusarium venenatum in deionised water) into the fermentation vessel 20. This gives a final fermentation media 10 volume of 13 L and an inoculum concentration of 7.7% v/v. Fermentation is carried out under a controlled aerobic environment at 30° C., with dissolved oxygen level (DO-30%) maintained using variable agitation (300 to 1200 rpm) and aeration (1 to 3 VVM). During fermentation, ammonium hydroxide (35%) is used for both pH control and as a source of nitrogen.

The fermentation is continued until a biomass (mycoprotein) concentration of approximately 18 g/L dry weight is achieved. The fermentation is then maintained by adding additional fresh fermentation media (outlined in Table 1) to the first fermentation vessel 20 at a rate equal to the growth rate of the microorganism (approximately 0.2 h⁻¹). The fresh fermentation media is added for approximately 3.5 hours to provide a final biomass (mycoprotein) concentration of approximately 20 g/L.

The resulting mixture comprising mycoprotein 60 and partially spent fermentation media 50 is removed from the fermentation vessel 20 for isolation, or separation 30.

In this experiment, the mycoprotein 60 and partially spent media 50 are separated using a disc stack centrifuge. However, it should be appreciated that other separation techniques may be used. For example, filtration or cross-flow filtration.

The mixture is fed into the disc stack centrifuge at a flow of approximately 10 L/h. The disc stack centrifuge was operated at a centrifugal force of approximately 10,000 g for 1 hour. The disc stack centrifuge used in this example is a GEA, Westfalia Pathfinder PSC 1 and the disc spacing was set at 0.77 mm.

During this step, the liquid phase is continuously collected from the centrifuge whilst the solids are captured within the bowl. Once the volume of solids within the bowl reaches the maximum capacity (1 L), the solid phase is ejected and collected as a slurry/paste. The slurry comprises mycoprotein 60 and partially spent fermentation media 50.

The total dry biomass collected from the disc stack separation is 127.8 g/L. The expected dry biomass from the disc stack separation is 169.4 g/L (calculated by multiplying the dry weight of the biomass in the mixture (18 g/L) with the volume of fermentation media fermented (9.41 L). Therefore, the solids recovery from the disc stack separation is 75%.

Under the conditions of this example, the volume of the mixture comprising mycoprotein and partially spent fermentation media was reduced by 92% by means of solids concentration using the described isolation, or dewatering step. The biomass concentration of the slurry/paste collected is approximately 18% (w/w) solids. However, this can be adjusted by changing the solids discharge interval or feed flow rate, depending on downstream process requirements with regards to solid/liquid content of the material taken forward through the downstream mycoprotein process steps.

A second disc stack separation can optionally be performed after the first disc stack separation. The remaining material (i.e., material remaining in the centrifuge after the first separation) is centrifuged at increasing flow rates of 20, 30 and 40 L/h. The aim of the second centrifugation is to force complete breakthrough of solids from the centrifuge.

The hyphal lengths of the mycoprotein filaments in the mixture were measured to determine whether the disc stack centrifuge would have an effect on the hyphal length.

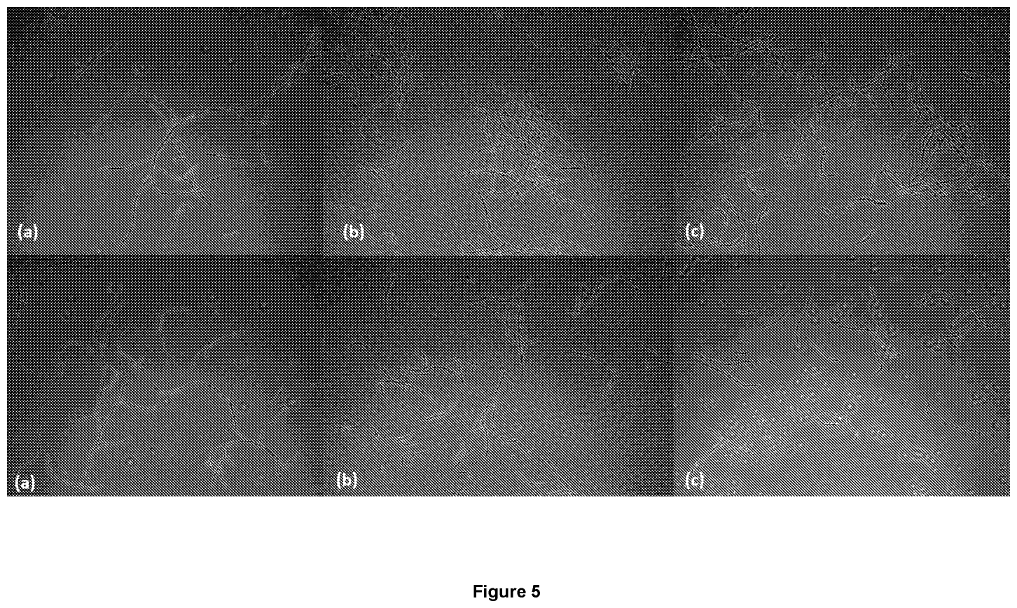

The mixture comprising mycoprotein and partially spent fermentation media was analysed using a scanning electron microscope (SEM) and the images produced are shown in FIG. 5. The images were captured using a Nikon Eclipse TE2000-S microscope in phage contrast mode and individual hyphal lengths were measured using Image J tracing software. Approximately 10 pictures per sample were captured and analysed to calculate the mean/median hyphal length for each sample.

FIG. 5 (a) shows two images of the mixture comprising mycoprotein 60 and partially spent fermentation media 50 obtained from the mycoprotein fermentation. Seven separate batches were analysed and the mean hyphal length is outlined in Table 2 below. FIG. 5 (b) shows two images of the solid-phase (mixture comprising mycoprotein 60 and partially spent fermentation media) recovered after a first disc stack separation. One batch was analysed and the mean hyphal length is outlined in Table 2 below. FIG. 5 (c) shows two images of the solid-phase (mixture comprising mycoprotein 60 and partially spent fermentation media) recovered after a second example of a first disc stack separation. One batch was analysed and the mean hyphal length is outlined in Table 2 below.

TABLE 2
Hyphal length measurements for: (i) mixture comprising
mycoprotein and partially spent fermentation media before isolation
(mycoprotein broth); and (ii) solid-phase recovered from disc stack
centrifugation.

| | Mean | SD | Median | |
|---|---|---|---|---|
| | Hyphal | Hyphal | Hyphal | No. of |
| | Length | Length | Length | measurements |
| Material | (μm) | (μm) | (μm) | taken |
| Mycoprotein broth - 1 | 346 | 219 | 298 | 97 |
| Mycoprotein broth - 2 | 368 | 271 | 297 | 80 |
| Mycoprotein broth - 3 | 381 | 288 | 282 | 89 |
| Mycoprotein broth - 4 | 802 | 472 | 768 | 41 |
| Mycoprotein broth - 5 | 516 | 362 | 444 | 39 |
| Mycoprotein broth - 6 | 490 | 326 | 423 | 61 |
| Mycoprotein broth - 7 | 295 | 175 | 234 | 72 |
| Solid-phase after first | 167 | 116 | 143 | 73 |
| separation - 1 | | | | |
| Solid-phase after first | 193 | 100 | 179 | 71 |
| separation - 2 | | | | |

Comparing mycoprotein broth-7 and the two centrifuged samples, a reduction in hyphal length of 39% and 30% for the first and second separation respectively were calculated. However, the reduction of 39% would still result in a mean hyphal length of >200 μm when applied to the other batches of mycoprotein broth.

Separation of the mixture prior to heat treatment is difficult using traditional separation techniques because the mixture is not gravity settling before heat treatment. However, this experiment shows that separation of the mixture comprising mycoprotein and partially spent fermentation media prior to heat treatment is feasible and use of the disc stack centrifuge maintains a reasonable hyphal length in the mycoprotein filaments.

The solid-phase is then taken through the remainder of the mycoprotein production process to isolate the final product.

The liquid-phase (isolated partially spent fermentation media) can be recycled back into the mycoprotein fermentation process by displacing the requirement for fresh water in the fermentation media 10. This step reduces raw material cost as the liquid-phase is nutrient rich and not degraded by heat treatment, and also reduces the costs and environmental load of treating the liquid-phase as waste effluent.

### Experiment 1B: Separation of Mycoprotein and Partially Spent Media Before Heat Treatment Step (18 L)

A further example as per Experiment 1A is carried out as follows.

A fermentation media 10 is prepared as per Table 1 for a working volume of 18 L. The fermentation media 10 is added to a 30 L fermentation vessel 20 and sterilised in situ at 121° C. for 30 minutes. At the end of the sterilisation cycle, the fermentation vessel 20 is cooled to 30° C., agitation set to 200 rpm and airflow to 1 VVM.

Biotin solution (0.000025 g/L) and choline hydrochloride solution (0.087 g/L) are filter sterilised and aseptically added to a heat sterilised glucose solution (autoclaved at 121° C. for 20 minutes). The glucose/biotin/choline solution is then aseptically transferred into the fermentation vessel 20.

The pH of the fermentation media 10 is then adjusted to pH 6.0 using 25% ammonium hydroxide solution, the DO probe is calibrated to 100% oxygen, and is set to control DO at 30% through cascade by stirrer then airflow.

The fermentation vessel 20 is inoculated with 10% working volume of Fusarium venenatum grown in seed culture to approximately 5 g/L biomass concentration.

The fermentation is operated in batch mode until the biomass reaches late exponential phase and the biomass (mycoprotein) concentration is constant at 12 g/L dry weight.

The resulting mixture comprising mycoprotein 60 and partially spent fermentation media 50 was harvested (removal of mycoprotein 60 and partially spent media 50) with the partially spent fermentation media 50 undergoing a dewatering step to separate a portion of the partially spent fermentation media 50, which is available for recycling back into the process.

The solids fraction from the dewatering step (comprising the mycoprotein) may be further processed with an RNA reduction step, and final solid liquid separation, to generate a 25% solid mycoprotein paste for use as a food ingredient.

A disc stack continuous centrifuge was used for the dewatering step in this example. The disc stack centrifuge was operated at a bowl speed of 10,000 g and a flow rate of 12 L/h. The disc stack bowl had a liquid hold-up volume of 1 L which was verified by pumping in water to fill the bowl and recording the volume added when flow appeared in the centrate line. The discharge interval was determined by amount of solids loaded into the bowl e.g. in 2 L of feed, 24 g of solids are added in total, by discharging after 2 L of feed added the 24 g will be discharged in 1 L volume providing a solids/centrate ratio of 2.4% resulting in dewatering of 50%.

Five examples were performed in this Experiment 1B, with each example aiming to vary the percentage of dewatering to demonstrate the degree of control and extent of dewatering that is feasible.

The aim of this Experiment 1B was to demonstrate dewatering of the partially spent fermentation media by 50%, which is illustrated in Experiment 1B, Example 2. In Example 2, twice the bowl capacity (2 L) was fed into the disc stack, and after 2 L of broth was added the solids in the bowl were discharged. At the point of discharge, the bowl theoretically contains 24 g of solids and 1 L of centrate, with the remaining 1 L having been continuously exiting the bowl as the centrates (low solids fraction). The material discharged from the bowl (thicks) was measured for percentage solids (dry solids) to calculate the percentage dewatering and concentration factor.

For the 50% dewatering (Experiment 1B, Example 2), the thicks fraction had a solids content of 2.34% (theoretical value=2.4%) and therefore dewatered the partially spent fermentation media by 49%. From an operational perspective, centrates removed can be returned to the fermentation vessel during a continuous fermentation without additional heat sterilisation.

Examples 1, 3, 4 and 5 were performed to demonstrate the dewatering step with either a reduced or increased target percent dewatering which may be applied during manufacturing depending on process requirements (see Table 3).

TABLE 3
Disc Stack Dewatering Examples 1-5 from Experiment 1B

| | Feed | Discharge | Thicks | Centrates | Concentration | % |
|---|---|---|---|---|---|---|
| Example | volume (L) | interval (min) | % solids | % solids | factor | Dewatering |
| 1 | 1.3 | 6 | 1.50 | 0.31 | 1.25 | 20 |
| 2 | 2 | 10 | 2.34 | 0.22 | 1.95 | 49 |
| 3 | 3 | 15 | 3.32 | 0.17 | 2.77 | 64 |
| 4 | 4 | 20 | 5.00 | 0.32 | 4.17 | 76 |
| 5 | 8 | 40 | 9.06 | 0.50 | 7.55 | 87 |

### Experiment 1C: Separation of Mycoprotein and Partially Spent Media Before Heat Treatment Step (100 L)

A further example as per Experiment 1A is carried out as follows.

A further fermentation media 10 was prepared as per Table 1 for a working volume of 100 L. A 300 L fermentation vessel 20 underwent steam sterilisation (SIP) prior to the fermentation media 10 (including glucose, biotin and choline) being filtered into the vessel.

The operating parameters (temperature, airflow, pressure, DO) were set up as per Experiments 1A and 1B. The fermentation vessel 20 was inoculated with 10 L of seed culture (Fusarium venenatum) at a biomass (mycoprotein) concentration of 14 g/L.

The fermentation vessel 20 was operated in batch mode until the biomass reached a concentration of 5 g/L. The resulting mixture comprising mycoprotein 60 and partially spent fermentation media 50 was harvested from the fermentation vessel 20 at a flow rate of 40 L/h, and it was fed into a decanter continuous centrifuge without heat treatment.

The decanter bowl speed and differential speed settings were altered across five examples (see Table 4) with the aim of dewatering the mixture to a solids content of ≤10% dry solids.

TABLE 4
Decanter Dewatering Examples 1 to 5 for Experiment 1C.

| | Bowl | Differential | | | |
|---|---|---|---|---|---|
| | speed | speed | Thicks | Concentration | % |
| Example | (rpm) | (rpm) | % solids | factor | Dewatering |
| 1 | 3000 | 200 | 15 | 30 | 97 |
| 2 | 3000 | 50 | 13.1 | 26.2 | 96 |
| 3 | 4000 | 150 | 12.5 | 25.0 | 96 |
| 4 | 4000 | 200 | 12.6 | 25.2 | 96 |
| 5 | 3000 | 125 | 10 | 20 | 95 |

Examples 1 to 5 were also subjected to heat shock (73° C., 20 minutes) in a water bath. This demonstrates the effectiveness of heat treatment in reducing total RNA content from an approximate value of 11% in fresh biomass to <2% as required for human consumption and to conform to the applicant's specification. The increased solids load (compared with 1.6% solids which is processed in the current manufacture of mycoprotein) will have an effect on heat transfer into the material. However, the data in Table 5 demonstrates sufficient RNA reduction in biomass solutions as dense as 12% total solids (equivalent to 120 g/L biomass concentration).

This level of dewatering prior to heat treatment will have the benefit of dramatically reducing the volume of material required to be heated to 73° C. during the RNA reduction step to produce a 25% solid paste as a final food ingredient.

TABLE 5
RNA Analysis Post Heat-treatment of Samples from Experiment 1C.

| % solids | % RNA |
|---|---|
| 0.93 | 1.44 |
| 7 | 0.70 |
| 8 | 0.92 |
| 10 | 1.16 |
| 12 | 0.59 |

### Experiment 2: Reintroducing at Least a Portion of the Partially Spent Media Separated after Heat Treatment into Mycoprotein Fermentation

Aerobic fermentation was performed to provide a mixture comprising mycoprotein 60 and partially spent fermentation media 50.

The mixture was subjected to heat treatment 40 in order to inactivate the microorganism, reduce the RNA content and kill any bacteria which may be present and which could interfere with subsequent process steps.

The mixture was separated by filtration to provide a solid-phase (mixture comprising mycoprotein 60 and partially spent fermentation media 50) and liquid-phase (isolated partially spent media 50). The filtration after heat treatment can be performed by any suitable filtration techniques. In this experiment, separation was by vacuum pump filtration using Whatman qualitative filter paper (Grade 4, 240 mm).

The isolated partially spent media 50 was then used as the water in a subsequent mycoprotein fermentation.

The subsequent mycoprotein fermentation was prepared as follows. A fermentation media 10 was prepared with the feedstock components in Table 6.

TABLE 6
Fermentation Media Components

| | Concentration |
|---|---|
| Component | (g/L) |
| Potassium dihydrogen phosphate (KH₂PO₄) | 20 |
| Potassium sulphate (K₂SO₄) | 0.3 |
| Ammonium chloride (NH₄Cl) | 4.4 |
| Magnesium sulphate, heptahydrate (MgSO₄•7H₂O) | 0.25 |
| Calcium chloride dehydrate (CaCl₂•2H₂O) | 0.01 |
| Iron (III) chloride hexahydrate (FeCl₃•6H₂O) | 0.01 |
| Citric acid (HOC(COOH)(CH₂COOH)₂•H₂O) | 0.0075 |
| Zinc chloride (ZnCl₂) | 0.005 |
| Manganese chloride dihydrate (MnCl₂•2H₂O) | 0.0041 |
| Copper chloride dihydrate (CuCl₂•2H₂O) | 0.001 |
| Cobalt chloride hexahydrate (CoCl₂•6H₂O) | 0.00145 |
| Sodium molybdate (Na₂MoO₄) | 0.001 |
| Biotin | 0.00005 |
| Glucose (pure) | 10 |

The components in Table 6 were added to a flask and made up to 1 L volume using the isolated partially spent media 50 from the first fermentation.

The media 10 comprising recycled isolated partially spent media 50 was fermented using Fusarium venenatum in a flask incubated in an orbital shaker at 30° C. and 150 rpm to provide a mixture comprising mycoprotein and partially spent fermentation media.

The aerobic fermentation reaction was analysed by HPLC at time intervals of 0, 4, 8, 20 and 24 hours after initiating the fermentation reaction. The pH of the reaction was monitored using a pH probe (Mettler-Toledo, U.K.) that was calibrated using buffers (pH 7 and pH 4). HPLC was performed using an Agilent 1290 Infinity LC System with a Rezex™ ROA-Organic Acid H+(8%), LC Column (150×7.8 mm). The HPLC conditions were as follows: the temperature of the column was maintained at 40° C., the mobile phase was 0.005 N sulfuric acid with a flow rate of 0.5 mL/min.

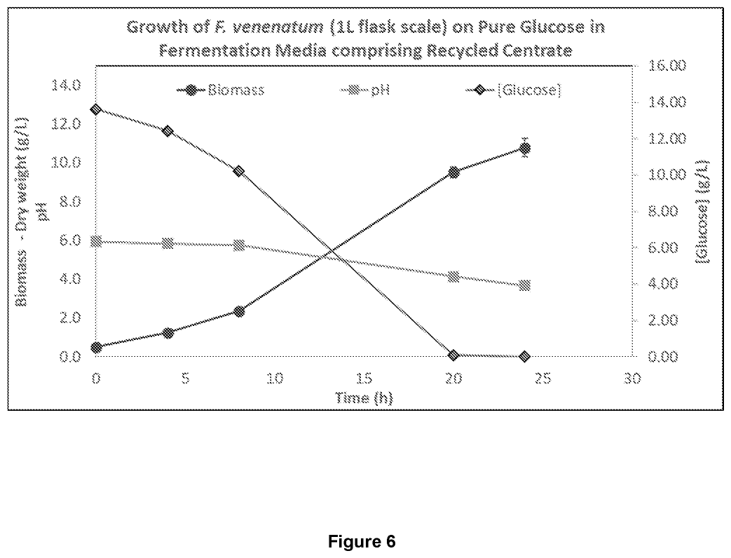

The experiment was conducted in duplicate and the results are provided in FIG. 6. Approximately 11 g/L of biomass (mycoprotein) was produced in the first 24 hours and the glucose concentration reduced from approximately 13 g/L to 0 g/L. The pH remained at approximately pH 6 for the first 8 hours and then reduced to pH 4 by 24 hours.

The biomass yield was calculated by dividing the change in biomass concentration over 24 hours by the change in glucose concentration over 24 hours. Using this calculation, the biomass yield was 0.75 Yx/s.

The biomass yield for a mycoprotein fermentation using distilled water (i.e., not using the recycled isolated partially spent media) was approximately 0.5 Yx/s (data not shown).

Therefore, this experiment shows that the biomass yield of the mycoprotein fermentation is not affected by the replacement of distilled water for recycled isolated partially spent media 50 in the fermentation media 10. A slightly higher biomass yield was obtained using isolated partially spent media. Without wishing to be bound by theory, this may be due to the microorganism being able to metabolise other products, such as amino acids, present in the partially spent media 50.

### Experiment 3: Control of Partially Spent Media Recycle Stream

A continuous aerobic fermentation was carried out using the fermentation media 10 and conditions outlined in Experiment 1A.

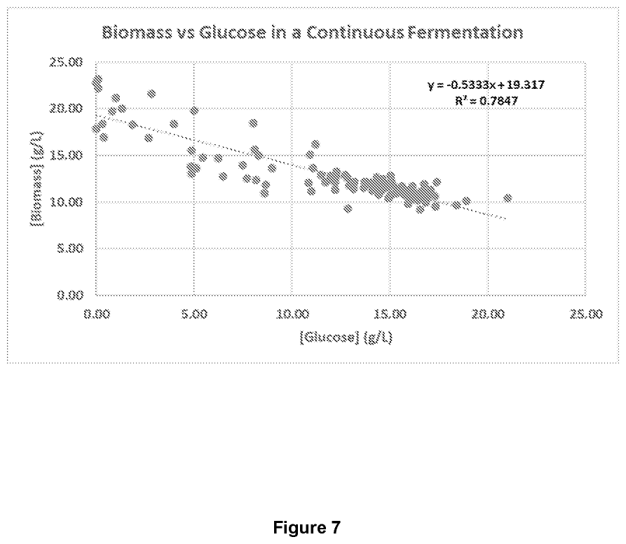

FIG. 7 shows a correlation graph of the biomass concentration versus the glucose concentration in the continuous fermentation reaction. This correlation can be used to extrapolate the glucose concentration from biomass readings during the fermentation reaction.

This can then be used to determine the volume of recycled isolated partially spent media and fermentation media required for the fermentation reaction. This theoretical experiment is based on the control of glucose concentration, but it should be appreciated that the same calculations can be done based on the control of any other component of the fermentation media.

The data in Table 7 was used to calculate the volume of isolated partially spent media and volume of fermentation media required for a mycoprotein fermentation using 50% recycle of isolated partially spent media in the process.

| | |
|---|---|
| Glucose concentration in glucose nutrient feed (g/L) | 700 |
| Glucose concentration in partially spent media (g/L) | 5 |
| Continuous feed of glucose required in the fermentation | 33 |
| vessel (g/L) | |
| Fermentation volume (L) | 150000 |
| Dilution rate (h⁻¹) | 0.2 |
| Feed flow rate (L/h) | 30000 |

Table 7: Data Used to Calculate Volume of Partially Spent Media and Volume of Fermentation Media Required for a Mycoprotein Fermentation Using 50% Recycle of Isolated Partially Spent Media.

Using the data in Table 7, the required fermentation media and partially spent media volume and concentration required to maintain a continuous feed of 33 g/L glucose into the fermentation vessel can be calculated.

The results are provided in Table 8 below.

TABLE 8
Volume of partially spent media and volume of fermentation
media required for a mycoprotein fermentation using 50%
recycle of partially spent media.

| | |
|---|---|
| Glucose concentration from nutrient feed in fermentation | 30.5 |
| media (g/L) | |
| Volume of nutrient feed in fermentation media (L) | 1307 |
| Volume of water in fermentation media (L) | 13693 |
| Glucose concentration from partially spent media (g/L) | 2.5 |
| Volume of partially spent media (L) | 15000 |

This shows that by introducing the recycle step the volume of water in the fermentation media can be reduced by approximately 15,000 L. This results in significant cost and energy savings for the mycoprotein production process.

When the mycoprotein production process is integrated with an ethanol biorefinery the cost savings are more significant due to the larger scale of the plant. For example, in a typical integrated process 48,000 kg/hr of water is provided from the biorefinery for the fermentation media 10 in the aerobic fermentation vessel 20. After the mycoprotein production process, the volume of water returned to the ethanol biorefinery to undergo anaerobic fermentation is 46770 kg/hr. However, by recycling the partially spent media from the mycoprotein fermentation process, the water required from the biorefinery as fermentation media 10 is reduced to 24000 kg/hr. Furthermore, the volume of water returned to the ethanol biorefinery to undergo anaerobic fermentation is reduced to 23,150 kg/hr. This reduces the effect on the bioethanol production process caused by the increased water input at the anaerobic fermentation stage and also reduces the waste effluent discharged from the process.

### Experiment 4: Determination of the Effect of Continuous Recycling of Partially Spent Media on Media Nutrients

A fermentation media 10 is prepared by adding the nutrients outlined in Table 9 to deionised water.

TABLE 9
Nutrient Composition of fermentation media

| Fermentation Media Component | Concentration (g/L) |
|---|---|
| Ammonium phosphate | 2.1 |
| (NH₄H₂PO₄) | |
| Potassium sulphate | 2.1 |
| (K₂SO₄) | |
| Magnesium sulphate heptahydrate | 0.87 |
| (MgSO₄•7H₂O) | |
| Calcium Acetate | 0.2 |
| (Ca(C₂H₃O₂)₂) | |
| Iron (II) sulphate heptahydrate | 0.00364 |
| (FeSO₄•7H₂O) | |
| Zinc sulphate heptahydrate | 0.0193 |
| (ZnSO₄•7H₂O) | |
| Manganese sulphate tetrahydrate | 0.01546 |
| (MnSO₄•4H₂O) | |
| Copper sulphate heptahydrate | 0.00185 |
| (CuSO₄•7H₂O) | |
| Phosphoric Acid | 3 (mL/L) |
| (85%) | |

The media 10 is added to a first fermentation vessel 20 and sterilised by heat-sterilisation using the introduction of steam to a heat jacket surrounding the fermentation vessel 20. The temperature is maintained at 121° C. for 30 minutes. Before sterilisation, care is taken to carefully secure all connections in the first fermentation vessel 20; for example, all addition ports are secured using rubber septum and respective collar fittings.

After sterilisation, filter sterilised glucose (44 g/L), biotin (0.00005 g/L), trace salts (Fe, Cu, Mn, Zn) and choline hydrochloride (0.087 g/L) are transferred into the first fermentation vessel 20 under aseptic conditions using a peristatic pump after the first fermentation vessel 20 is cooled down to an ambient temperature.

A dissolved oxygen (DO) probe is inserted into the fermentation vessel 20 before sterilisation. The probe is then calibrated after sterilisation. The DO probe is calibrated at a fermentation temperature of 30° C., with an air flow of 10 L/min (1 VVM) and stirring speed of 300 rpm using compressed air and nitrogen gas. Nitrogen gas is flushed through a sparger at a rate of 10 L/min to achieve 0% calibration of the DO probe. Similarly, compressed air is then sparged into the fermentation media 10 until saturation is achieved (i.e., a constant reading is observed) to allow 100% calibration. The air enters the first fermentation vessel 20 through a sterile inlet filter and sparger. Air escapes first through a condenser, to ensure there is no loss of media 10, and then through an exit filter. Thereafter, the pH of the fermentation media 10 is adjusted to pH 6.0 using a suitable base (in this example 35% Ammonium Hydroxide is used as the base).

Fermentation is initiated by adding 1 L of 1% w/v inoculum (Fusarium venenatum in deionised water) into the fermentation vessel 20. This provides a final concentration of 10% v/v inoculum in the fermentation vessel. Fermentation is carried out under a controlled aerobic environment at 30° C., with dissolved oxygen level (DO-30%) maintained using variable agitation (300 to 1200 rpm) and aeration (1 to 3 VVM). During fermentation, ammonium hydroxide (35%) is used for both pH control and as a source of nitrogen.

After fermentation is complete, the mixture comprising mycoprotein and partially spent fermentation media is subjected to heat treatment in order to inactivate the microorganism and reduce the RNA content.

The mixture is separated by filtration to provide a solid-phase (mycoprotein 60 and partially spent fermentation media 50) and liquid-phase (isolated partially spent media 50). The filtration after heat treatment can be performed by any suitable filtration techniques. In this experiment, separation was by vacuum pump filtration using Whatman qualitative filter paper (Grade 4, 240 mm).

The isolated partially spent media 50 is then filter sterilised and used as the water in the media of a subsequent mycoprotein fermentation.

This experiment was repeated six times to determine the effect of media recycling on nutrients in the fermentation media.

Specifically, the first batch was prepared as outlined above and the concentration of potassium, sulphate, phosphate, calcium, magnesium and glucose in the mixture comprising mycoprotein 60 and partially spent media 50 was measured at the end of fermentation after the heat treatment step.

The second batch was prepared by substituting 5 L of the water in the fermentation media with 5 L of isolated partially spent media 50 from the first batch. The isolated partially spent media is filter sterilised before being introduced to the second batch. All other media components for the fermentation media in the second batch were the same as for the first batch.

The next four batches were prepared in a similar manner, with 5 L of isolated partially spent media 50 from the previous batch being added to each subsequent batch.

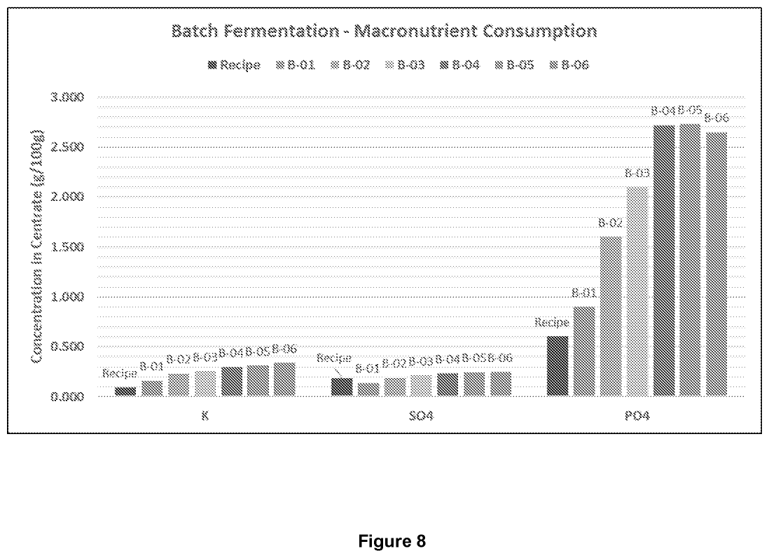

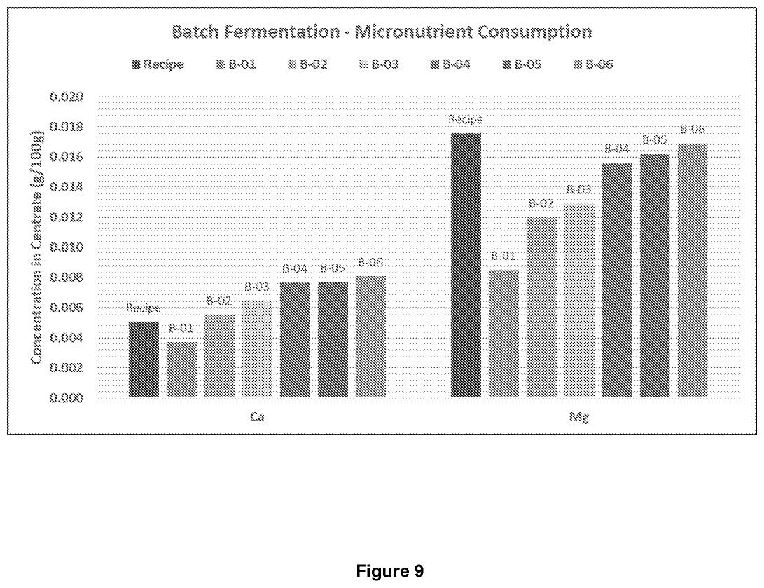

The results are shown in FIGS. 8 and 9. In particular, the data shows that the media nutrients reach an equilibrium after approximately four subsequent fermentations. Magnesium is the media component that is consumed the most during fermentation and could be used to determine a recycling strategy as outlined in Experiment 3. Furthermore, it was found that the accumulation of nutrients in the recycled isolated partially fermented media has no detrimental effect on the microorganism or mycoprotein product.

### Experiment 5: Recycling Dewatered Partially Fermented Media

A number of continuous fermentations were undertaken in 10 L, 150 L and 200 L stirred tank reactors (fermentation vessels) with a fermentation media composition as shown in Table 10 and using the conditions and procedure outlined in Experiment 1A. In use, and when partially spent fermentation media is combined with original fermentation media, the so-formed continuous fermentation media comprises the components as outlined in Table 10. The components comprise glucose and nutrients. A source of nitrogen is also required and is added under a separate independent control.

TABLE 10
Fermentation Media
Amounts of Components in Fermentation Media and/or Continuous Fermentation Media
(Partially Spent Fermentation Media and Original Fermentation Media)

| | Lower | Lower Middle | Typical | Upper Middle | Upper |
|---|---|---|---|---|---|
| | Concentration | Concentration | Concentration | Concentration | Concentration |
| Chemical | (g/L) | (g/L) | (g/L) | (g/L) | (g/L) |
| K₂SO₄ | 1 | 1.5 | 2 | 2.5 | 3 |
| MgSO₄•7H₂O | 0.45 | 0.68 | 0.9 | 1.13 | 1.35 |
| Ca(C₂H₃O₂)₂ | 0.1 | 0.15 | 0.2 | 0.25 | 0.3 |
| Phosphoric | 0.575 | 0.86 | 1.15 | 1.44 | 1.725 |
| acid (85%) | | | | | |
| FeSO₄•7H₂O | 0.0025 | 0.004 | 0.005 | 0.006 | 0.0075 |
| ZnSO₄•7H₂O | 0.0125 | 0.019 | 0.025 | 0.031 | 0.0375 |
| MnSO₄•4H₂O | 0.01 | 0.015 | 0.02 | 0.025 | 0.03 |
| CuSO₄•5H₂O | 0.00125 | 0.0019 | 0.0025 | 0.0031 | 0.00375 |
| Biotin | 0.0000125 | 0.000019 | 0.000025 | 0.000031 | 0.0000375 |
| Glucose | 16.5 | 15 | 33 | 44 | 49.5 |
| Choline | 0.0435 | 0.065 | 0.087 | 0.109 | 0.1305 |
| Hydrochloride | | | | | |

In the experiments outlined herein at least a portion of the partially spent fermentation media is isolated and then reintroduced to the fermentation vessel, thus mixing with original fermentation media and forming a continuous fermentation media. When this is carried out, the amount of carbohydrate (for example, glucose) is maintained at a concentration of at least 15 g/L, optionally 33 g/L in the fermentation media prior to fermentation. Other nutrients are maintained within the concentration ranges stated in Table 10 in the fermentation media prior to fermentation. A nitrogen source (e.g., ammonium hydroxide, urea, gaseous ammonia) is added and is controlled separately to the carbohydrate and other nutrient feed.

The fermentation conditions as well as the fermentation media were similar for the experiments outlined herein. The dilution rate during the continuous phase was adjusted to match the specific growth rate of the mycoprotein organism, which varied between 0.17 h⁻¹ and 0.2 h⁻¹. The flow of the continuous media into the fermentation vessel and the harvest of fermentation broth are controlled to match the growth rate of the organism (i.e., between 0.17 h⁻¹ and 0.2 h⁻¹). The vessel volume multiplied by the growth rate=the dilution rate (L/h).

Centrate samples were taken at various timepoints throughout these steady state continuous fermentations and the biomass concentration within the fermentation vessel noted (via dry cell weight measurements of fermentation media) at each timepoint. The centrate samples were taken from the liquid stream after centrifugation (e.g., post decanter centrifuge).

A correlation was then estimated and a Yield coefficient (Yx/s=Yield of biomass per unit mass of substrate) calculated for glucose and for each nutrient element or ion. This is illustrated in Table 11.

TABLE 11
Yield Coefficients for Biomass

| Name of Component in | Yield Coefficient (Yx/s [g/g]) | |
|---|---|---|
| Fermentation Media | Minimum | Maximum |
| K | 15 | 45 |
| P | 25 | 175 |
| SO₄ | 5 | 100 |
| Ca | 350 | 1,100 |
| Mg | 400 | 1,200 |
| Zn | 2,000 | 14,000 |
| Fe | 3,400 | 10,000 |
| Cu | 24,000 | 74,000 |
| Mn | 725 | 6,000 |
| Glucose | 0.2 | 0.85 |

These values from Table 11 in combination with a known biomass concentration in the fermentation vessel at any given point in time can be used to maintain the correct amount of glucose (or other nutrients) in the continuous fermentation media. For example, using data of glucose utilisation, which allows a user to calculate the expected media composition of centrates exiting a fermentation vessel containing a given biomass concentration, the amount or the components of the original unfermented fermentation media being fed into the continuous fermentation can be adjusted to the correct composition whereby, when combined with the at least partially spent fermentation media, the working concentration of all media components mentioned in Table 10 remains constant.

In more detail, using the values from Tables 10 and 11, and knowing at least some of the variables (e.g., biomass concentration, specific growth rate of the organism and/or working volume of the fermentation vessel), the expected concentration of a particular component of the partially spent fermentation media can be calculated. In turn, this can be used to calculate the balance or amendment that should be made to the original (i.e., not previously fermented) fermentation media to ensure that the components of the continuous fermentation media (i.e., comprising and/or containing both partially spent fermentation media and original fermentation media) remain within the ranges given in Table 10, for example.

As noted, the expected consumption of each ion species can be calculated and then the original fermentation media (“fresh” feed) going into the fermentation vessel can be balanced with the recycled partially spent fermentation media also going into the fermentation vessel by reducing or otherwise amending the amount or the components of the original fermentation media.

The equation dictating this is:

$$B {Q}_{f} - ( \frac{1}{A} ) X μ V = C {Q}_{f}$$

### Where:

A=Yield coefficient (as stated above) (g/g)
B=working concentration of “fresh” feed (g/L)
C=working concentration of “recycle” feed stream (g/L)
X=dry cell weight of biomass in the fermenter (g/L)
μ=growth rate of organism (h⁻¹)
V=working volume of the fermenter
Q_{f}=volumetric flow rate of recycle (L/h)

The term on the right-hand side can be used to adjust the concentration of ‘B’ when recycling starts.

The proportion of at least partially spent fermentation media being returned to the fermentation vessel can vary from 1% by volume (of the total amount of partially spent fermentation media) to a maximum of 95% by volume, but typically is 40% to 60% by volume, and most typically is 50% by volume.

In this way a significant proportion of fresh water usage is removed from the process and “waste” water and unused glucose, source of nitrogen and nutrients (that are in the at least partially spent fermentation media) can be recycled rather than being passed to a second fermentation (such as a yeast fermentation for the production of alcohol) or alternatively to a waste water treatment process.

The improved process as described herein provides an efficient, cost effective process for obtaining mycoprotein. The process can be incorporated into existing ethanol biorefineries and no additional chemicals or modifications to the existing process are required. Furthermore, the waste effluent discharged from the process is significantly reduced, volume of feedstock is reduced and processing times are decreased. This results in a more efficient, cost effective and environmentally friendly process for producing mycoprotein.

While this invention has been described with reference to the sample embodiments thereof, it will be appreciated by those of ordinary skill in the art that modifications can be made to the structure and elements of the invention without departing from the spirit and scope of the invention as a whole.

1. A continuous process for producing and isolating mycoprotein, the process comprising:
(i) providing a fermentation media suitable for producing mycoprotein;
(ii) introducing the fermentation media to a first fermentation vessel;
(iii) fermenting the fermentation media to obtain a mixture comprising mycoprotein and partially spent fermentation media;
(iv) isolating at least part of the partially spent fermentation media from the mixture comprising mycoprotein and partially spent fermentation media; and
(v) reintroducing at least a portion of the isolated partially spent fermentation media into the first fermentation vessel.

2. The process of claim 1, wherein the fermentation media comprises a carbohydrate suitable for producing mycoprotein.

3. The process of claim 2, wherein at least one of: (a) the provision of fermentation media after the reintroduction of a portion of the isolated partially spent fermentation media; and (b) the reintroduction of least a portion of the isolated partially spent fermentation media, is configured to maintain an excess of carbohydrate prior to fermentation.

4. The process of claim 3, wherein maintaining an excess of carbohydrate prior to fermentation comprises: (a) determining the concentration of carbohydrate in the partially spent fermentation media; and (b) adjusting the fermentation media introduced to the first fermentation vessel to maintain an excess of carbohydrate prior to fermentation.

5. The process of claim 1, wherein the first fermentation vessel is an aerobic fermentation vessel.

6. (canceled)

7. The process of claim 1, wherein the process comprises the additional step of heating the mixture comprising mycoprotein and partially spent fermentation media.

8. The process of claim 7, wherein the step of heating the mixture comprising mycoprotein and partially spent fermentation media is after the step of isolating at least part of the partially spent fermentation media from the mixture.

9. The process of claim 7, wherein the step of heating the mixture comprising mycoprotein and partially spent fermentation media is after the step of reintroducing at least a portion of the isolated partially spent fermentation media into the first fermentation vessel.

10. The process of claim 7, wherein the step of heating the mixture comprising mycoprotein and partially spent fermentation media is before the step of isolating at least part of the partially spent fermentation media from the mixture.

11. The process of claim 7, wherein the step of isolating at least part of the partially spent fermentation media from the mixture comprises a first isolation step and a second isolation step.

12. The process of claim 11, wherein the first isolation step is before the step of heating the mixture comprising mycoprotein and partially spent fermentation media and the second isolation step is after the step of heating the mixture comprising mycoprotein and partially spent fermentation media.

13. The process of claim 11, wherein the step of reintroducing at least a portion of the isolated partially spent fermentation media into the first fermentation vessel comprises a first reintroduction step and a second reintroduction step.

14. The process of claim 13, wherein the first reintroduction step is after the first isolation step and before the step of heating the mixture comprising mycoprotein and partially spent fermentation media.

15. The process of claim 13, wherein the second reintroduction step is after the second isolation step.

16. The process of claim 1, wherein the mycoprotein in the mixture is a substantially solid phase and the partially spent fermentation media in the mixture is a substantially liquid phase comprising nutrients and a carbohydrate, and wherein the step of isolating at least part of the partially spent fermentation media from the mixture comprising mycoprotein and partially spent fermentation media comprises separating the substantially solid phase and the substantially liquid phase.

17. The process of claim 16, wherein the step of isolating at least part of the partially spent fermentation media from the mixture comprising mycoprotein and partially spent fermentation media comprises separating the substantially solid phase and the substantially liquid phase by centrifugation.

18. The process of claim 1, wherein the reintroduction of at least a portion of the partially spent fermentation media into the first fermentation vessel is a recycle step, optionally wherein the reintroduction of at least a portion of the partially spent fermentation media into the first fermentation vessel decreases the amount of fermentation media required for the process.

19. The process of claim 1, wherein the process comprises the additional step of producing and isolating ethanol.

20. (canceled)

21. The process of claim 1, wherein the process comprises the additional step of introducing at least a portion of the isolated partially spent fermentation media into a second fermentation vessel.

22. The process of claim 21, wherein the step of introducing at least a portion of the isolated partially spent fermentation media into the second fermentation vessel is after the step of reintroducing at least a portion of the isolated partially spent fermentation media into the first fermentation vessel.

23. The process of claim 21, wherein the process comprises the additional step of fermenting the at least a portion of the isolated partially spent fermentation media in the second fermentation vessel to obtain ethanol.

24. The process of claim 21, wherein at least a portion of the isolated partially spent fermentation media is reintroduced into the first fermentation vessel and a remainder of the isolated partially spent fermentation media is introduced into the second fermentation vessel.

25. Mycoprotein obtainable, obtained or directly obtained by the process of claim 1.

26. The process of claim 4, wherein adjusting the fermentation media comprises reducing at least one of: the amount of fermentation media provided; and the concentration of carbohydrate therein.

27. The process of claim 12, wherein the step of reintroducing at least a portion of the isolated partially spent fermentation media into the first fermentation vessel comprises a first reintroduction step and a second reintroduction step.
